(19) United States
(12) Patent Application Publication
(10) Pub. No.: US 2018/0028473 A1
(43) Pub. Date: Feb. 1, 2018

(54) NUTRIENTS SOLUTIONS

(71) Applicant: Mitochondrial Substrate Invention Ltd., London (GB)

(72) Inventors: Igor Anatolievich POMYTKIN, London (GB); Anton CHERNOPIATKO, London (GB); Larisa ANDREEVA, London (GB)

(73) Assignee: Mitochondrial Substrate Invention Ltd., London (GB)

(21) Appl. No.: 15/729,516

(22) Filed: Oct. 10, 2017

Related U.S. Application Data
(63) Continuation-in-part of application No. 14/753,304, filed on Jun. 29, 2015.

(30) Foreign Application Priority Data
Jun. 30, 2014 (GB) 1411570.3
Jul. 11, 2014 (GB) 1412414.3

(51) Int. Cl. A61K 31/14; A23L 2/52; A61K 33/00

(52) U.S. Cl. CPC A61K 31/14; A61K 33/00; A23L 2/52; A23V 2002/00

(57) ABSTRACT

The present invention relates to dietary supplements, medical foods, and pharmaceutical compositions comprising an aqueous solution of salt of choline, wherein solution comprises a deuterium depleted water having content of deuterium from about 90 to about 135 ppm and ¹H₂¹⁶O isotopologue up to 99.759%, in particular, the invention relates to an aqueous solution of succinate salt of choline, wherein the aqueous solution comprises said deuterium depleted water.

### CROSS-REFERENCE TO RELATED APPLICATIONS

This application is a continuation-in-part of application Ser. No. 14/753,304, filed on Jun. 29, 2015, which is incorporated herein in its entirety. Priority is also claimed to GB 1412414.3, filed Jul. 11, 2014, and to GB 1411570.3, filed Jun. 30, 2014.

### FIELD OF THE INVENTION

The present invention relates to nutritional compositions. In particular, the invention relates to nutritional compositions comprising a consumable choline formulation.

### BACKGROUND OF THE INVENTION

Choline is an essential nutrient for healthy metabolic functioning. Choline is needed for biosynthesis of acetylcholine, neurotransmitter crucial for communication of neurons in the nervous system. Choline deficiency leads to neurological disorders (Zeisel S H et al, Nutr Rev. 2009,67(11):615-623), and oral administration of choline enhances cognitive function in relatively impaired performers (Knott V et al., Pharmacology, Biochemistry and Behavior 2015, 131:119-129). An adequate intake level for choline is 550 mg/day for men and 425 mg/day for women. The de novo choline synthesis is not sufficient to meet human requirements and choline must be obtained through the diet containing choline-rich foods, like eggs and shrimps, or as nutritional addition to a normal diet, for example in form of choline salts. Choline salts, e.g. choline citrate, choline bitartrate, choline succinate (2:1) salt, and choline chloride are commercially available and contain 21%, 41%, 64%, and 74% of choline, respectively. However, despite of numerous research results strongly indicate that choline salts ingestion enhances memory of rodents (see e.g. Meck W. H & Williams C. L., 2003, Neurosci Biobehav Rev 27:385-399), data regarding effects choline salts on memory and, especially, on cognition of humans are still incomplete. Further, in some studies it has been demonstrated that the effects may vary depending on the choline salt species used in the study and test group of individuals: from no to significant effects in healthy, impaired or low base-line performers. For example, it has been shown that choline bitartrate does not boost memory performance and learning of healthy participants (Lippelt et al., 2016, PLoS ONE 11(6)), however, recently, two studies by Knott et al. (Pharmacol Biochem Behay. 2015, 131:119-29; and Neurosci Lett. 2015, 591:121-5) demonstrated that not only impaired participants with low baseline performance, but also healthy participants benefit CDP-choline supplementation for improvement of cognitive function on a variety of tasks; choline alphoscerate worked also well as cognition enhancing agent in a study by Traini et al. (Traini et al., 2013, Current Alzheimer Research 10:1070-1079). Choline succinate (2:1) salt has been shown to have cognition enhancing properties in rodents (US2006/0199862A1), and, also, that it is capable to enhance insulin receptor activation in neurons (Storozheva et al., BMC Pharmacol. 2008, 8:1-13). Activation of neuronal insulin receptor is documented to take place in the processes associated with cognition and memory (see e.g. Lee S-H et al., 2016, Molecular Metabolism 5(8):589-601). Accordingly, salts choline, and especially choline succinate (2:1), seem to have superior nutritional value, e.g. as food supplements with the potential to enhance cognition and memory.

To provide fast delivery of choline to a human body, choline supplements are frequently formulated as a beverage. However, all known choline salts give a specific unpleasant taste to the beverage, unacceptable to a consumer, when taken in amounts recommended by FDA (≧55 mg of choline per serving). This common disadvantage generally limits the use of choline salts as ingredients in beverages. Therefore, it would be a great advantage that a consumable choline formulation, e.g. a beverage, does not have this unpleasant taste.

Commercial beverages typically contain natural water, which is a composition of nine water isotopologues (¹H₂¹⁶O, ¹H₂¹⁷O, ¹H₂¹⁸O, ¹H¹⁶O²H, ¹H¹⁷O²H, ¹H¹⁸O²H, ²H₂¹⁶O, ²H₂¹⁷O, ²H₂¹⁸O) formed by stable isotopes of hydrogen ¹H and ²H (D, deuterium) and oxygen (¹⁶O, ¹⁷O, and ¹⁸O). The sum of fractional abundances of four major isotopologues ¹H₂¹⁶O (i.e. H₂O), ¹H₂¹⁸O, ¹H₂¹⁷O, and ¹H¹⁶O²H (i.e. HOD) is 0.99999952, wherein the individual abundances are as follows: 0.997317 (H₂O); 0.00199983 (¹H₂¹⁸O); 0.000372 (¹H₂¹⁷O); 0.00031069 (HOD). Rothman LS et al, J Quantitative Spectroscopy &. Radiative Transfer 2003, 82:9. Natural abundances of the rest five deuterium-bearing isotopologues (¹H¹⁷O²H, ¹H¹⁸O²H, ²H₂¹⁶O, ²H₂¹⁷O, and ²H₂¹⁸O) are too small to be measured by currently available methods. Thus, deuterium is completely incorporated in HOD isotopologue, which content in natural water is 0.031069 mol. %.

The deuterium content in water samples is measured by isotope ratio mass-spectrometry and expressed as deuterium-to-protium ratio R=D/H, in ppm units, where D is the number of deuterium atoms, and H is the number of protium atoms. Ocean water contains deuterium at level of 155 ppm (Vienna Standard Mean Ocean Water 2, VSMOW2). Continental waters slightly differ from ocean water in deuterium content, since isotope fractionation occurs during water evaporation-precipitation process in nature. Majority of people reside at places, where they consume water having deuterium content from 140 to 155 ppm. (Kendall et al, Hydrol Processes 2001, 15(7):1363-1393. Bowen et al, Water Resour Res 2007, 43, W03419).

Deuterium depleted water (DDW) has been shown to have a variety of biological effects. Still, the mechanism of biological effects of DDW remains to be unknown and data on the effects are rather inconsistent, e.g. Bild et al (Rom J Physiol, 2004 41(1-2):53-67) showed that DDW having 30 ppm deuterium has a stimulatory effect on both neoplastic and normal cell proliferation in vitro, which is in contrary to the data by Somlayi et al (J of Oncol 1998. 30(4):91-94)) and Wang et al, (Biomed Pharmacother. 2013, 67(6):489-96) that showed inhibitory effects of DDW on cancer cell proliferation and expression of certain oncogenes, correspondingly. There has been also described the DDW effect of reduction of blood glucose levels in diabetic rats, where the maximum of the effect was observed at 130 ppm deuterium, while DDW with higher or lower deuterium content was much less efficient (Molnar M et al., 2012 http://www.deuteriumdepletion.com/2012program.php). Further, there has been also reported that DDW having 5 ppm content of deuterium has enhancing cognition effect in mice (RU 2338542). In other studies, it was demonstrated that DDW has a synergetic effect with some anti-cancer drugs, e.g. in study by Krempels et al, (Integrative Cancer Therapies 2008, 7(3): 172-181) it was demonstrated a prolonged survival time of lung cancer patients with brain metastases that received the cisplatinum-etoposide chemotherapy combined with DDW (10-20 ppm deuterium) treatment, and in study by Soleyman-Jahi et al. (As Pac J Cancer Res 2014 15(5):21-79-2183) it was demonstrated that the cytotoxic effect of pacliitaxel on cancer cells in vitro may be strengthen by DDW having 40 ppm, 62 ppm or 89 ppm deuterium.

However, despite of treatment with DDW has been demonstrated to have positive effects in connection with a variety of symptoms and diseases, there is still lack of validation studies and inconsistency of research data, such as e.g. a non-linear correlation between the deuterium content and the scale of an effect (Cong et al. Experimental and therapeutic medicine 2010, 1:277-83), or significant variation of the effect in different cell types (Soleyman-Jahi et al 2014 see above). Accordingly, it would be advantageous to provide compositions comprising DDW with a certain content of deuterium that are proven to have a desirable biological effect, especially when said DDW is intended as adjuvant or solvent for another biologically active substance.

### BRIEF DESCRIPTION OF THE DRAWING

FIG. 1 shows the scheme of the process for preparing water having deuterium content from about 90 to about 135 ppm and up to 99.759% of isotopologue 1H₂¹⁶O.

FIG. 2 shows the effects of the aqueous solution of the present invention on retention latencies in rats with scopolamine-induced amnesia during the second session of the retention trial.

### DETAILED DESCRIPTION OF THE INVENTION

The present invention provides an aqueous choline salt formulation that not only retains all biologically beneficial features of said choline salt consumed as a nutritional supplement, but also lacks some its disadvantages, e.g an unpleasant taste which limits commercial usability of the salt. It is surprisingly found that a solution of a choline salt, in particular choline succinate (2:1), in deuterium depleted water (DDW) that has content of deuterium in the range from about 90 to about 135 ppm and ¹H₂¹⁶O isotopologue up to 99.759% has a pleasant taste when consumed as a drink, compared to a solution of this salt in normal drinking water having the content of deuterium around 155 ppm that has a typical unpleasant taste of an aqueous solution of choline (the term “about” in the present context means a variation in the concerned value equal to 1-3% of the value). Accordingly, a first aspect of the present invention relates to an aqueous solution of a choline salt, preferably choline succinate (2:1) salt, wherein the aqueous solution comprises DDW having content of deuterium from about 90 to about 135 ppm and ¹H₂¹⁶O isotopologue up to 99.759%. The aqueous solution in one embodiment is a beverage.

Further, surprisingly, the inventors found that an aqueous solution of choline succinate (2:1) salt comprising DDW that has content of deuterium from about 90 to about 135 ppm and ¹H₂¹⁶O isotopologue up to 99.759% has a significant stimulatory effect on cognition. Even more surprisingly, the stimulatory effect of choline succinate (2:1) dissolved in DDW of the invention, i.e. DDW that has content of deuterium from about 90 to about 135 ppm and ¹H₂¹⁶O isotopologue up to 99.759%, is higher compared to the effect of an aqueous solution of the salt in normal drinking water with the content of deuterium around 155 ppm, and said DDW per se does not have any stimulatory effect on cognition. Accordingly, in a second aspect, the invention provides a method for enhancing cognitive function in a subject, comprising a step of administering to the subject an effective amount of an aqueous solution of choline succinate (2:1), said aqueous solution comprising water having content of deuterium from about 90 to about 135 ppm and ¹H₂¹⁶O isotopologue up to 99.759%.

Further, the present invention relates to a digestible capsule comprising an aqueous solution of a choline salt, preferably choline succinate (2:1) salt, wherein said solution comprises a water having content of deuterium from about 90 to about 135 ppm and ¹H₂¹⁶O isotopologue up to 99.759%.

As used herein, the term “water” refers to a composition of water isotopologues formed by stable isotopes of hydrogen (¹H and ²H) and oxygen (¹⁶O , ¹⁷O and ¹⁸O).

As used herein, the term “isotopologue” is in accordance with IUPAC Compendium of Chemical Terminology 2nd Edition (1997) and refers to a molecular entity that differs only in isotopic composition (number of isotopic substitutions). Examples of water isotopologues include ¹ H₂¹⁶O (i.e. H₂O), ¹H₂¹⁷O, ¹H₂¹⁸O, ¹H¹⁶O²H (i.e. HOD), ¹H¹⁷O²H, ¹H¹⁸O²H, ²H₂¹⁶O, ²H₂¹⁷O, and ²H₂¹⁸O.

As used herein, the term “deuterium content” refers to the amount of deuterium incorporated in water and expressed as ratio R=D/H, in ppm units, where D is the number of deuterium atoms, and H is the number of protium atoms. In practicing the invention, the deuterium content of water is from about 90 to about 135 ppm; wherein term “about” means 1-3% of the value, e.g. “about 135 ppm” means 135±4 ppm.

In practicing the invention, the water having deuterium content from about 90 to about 135 ppm and 99.759% or less of isotopologue ¹H₂¹⁶O can be prepared by any process comprising a step of reducing the deuterium content in the natural water including, but are not limited to, a vacuum distillation of natural water.

In practicing the invention, the deuterium content can be measured by isotope ratio mass-spectrometry and expressed as ratio R=D/H, in ppm units, where D is the number of deuterium atoms, and H is the number of protium atoms.

As used herein, the term “aqueous solution” refers to any system having a water level of more than 40 wt. %, preferably more than 50 wt. %.

As used herein the term “choline salt” refers to any salt of choline suitable for consumption by a human. Non-limiting examples of choline salts of the invention include choline citrate (CAS No.546-63-4), choline bitartrate CAS No. 87-67-2, choline succinate (2:1) (CAS Number: 71-27-2), and choline chloride (CAS No. 67-48-1). Preferably, the choline salt is choline succinate (2:1), which is defined by formula (I) below:

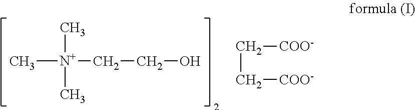

Choline succinate 2:1 salt is interchangeably termed herein as “compound of formula (1)”.

As used herein, the term “cognitive function” refers to any intellectual brain process involved in information processing and storage including, but are not limited to, attention, knowing, thinking, information processing, language, learning, sensing, reasoning, ability to memory, working memory, short-term memory, long-term memory, anterograde memory, retrograde memory, memory retrieval, decision-making, action, problem solving, and mental imagery.

As used herein, the term “enhancing cognitive function” refers to enhancing any aspect of intellectual brain process involved in information processing and storage including, but are not limited to, attention, knowing, thinking, information processing, language, learning, sensing, reasoning, ability to memory, working memory, short-term memory, long-term memory, anterograde memory, retrograde memory, memory retrieval, decision-making, action, problem solving, and mental imagery.

Efficacy of an agent to enhance cognitive function can be assessed by conventional methods. In humans, the efficacy can be assessed by standardized tools, such as modified mini-mental state exam, Folstein mini-mental state examination, short portable mental status questionnaire, and Alzheimer's disease assessment scale. In animals, the enhancement of cognitive function can be assessed using well-known behavior tests such as passive avoidance test, Morris water maze test, novel cage test, and modified elevated plus-maze test. Itoh J et al, Psychopharmacology 19about 90, 101:27-33. ltoh J et al, Eur J Pharmacol 1991, 194:71-76. Wultsch T et al, J Neural Transm. 2007, [Suppl 72]: 69-85, p.71. Accordingly, the effect of the aqueous solution of the invention on cognitive function can be assessed by methods well-known from the art including, but are not limited to, standardized tools such as Modified Mini-Mental State Exam, Folstein Mini-Mental State Examination, Short Portable Mental Status Questionnaire, Alzheimer's Disease Assessment Scale, Clinical Dementia Rating, Clock Drawing Test, Neuropsychiatric Inventory, Middlesex Elderly Assessment of Mental State or any similarly designed test. Instrumental measures of neuron functioning may also be used for the assessment of cognitive function including, but are not limited to, ¹H and ³¹P Nuclear Magnetic Resonance spectroscopy (NMR), Magnetic Resonance Imaging (MRI); Functional Magnetic Resonance Imaging (fMRI);Computed Tomography (CT); Computed Axial Tomography (CAT); Positron Emission Tomography (PET): Single Photon Emission Computed Tomography (SPECT); Diffuse Optical Imaging (DOI); Diffuse Optical Tomography (DOT); Z-score on the voxel-based specific regional analysis system for Alzheimer's disease (VSRAD); Exploratory eye movements recording; Event-related potentials (EPR) recording; or any similarly designed instrumentation. Using such tests, a skilled clinician would be able to assess the level of cognitive deficit of a patient or enhanced cognitive function following treatment.

As used herein, the term “cognitive deficit” refers to impairment of any aspect of intellectual brain process involved in information processing and storage including, but are not limited to, attention, knowing, thinking, information processing, language, learning, sensing, reasoning, ability to memory, working memory, short-term memory, long-term memory, anterograde memory, retrograde memory, memory retrieval, decision-making, action, problem solving, and mental imagery.

Preferably, the cognitive deficit is selected from a group consisting of age-related cognitive impairment and mild cognitive impairment.

Preferably, the cognitive deficit is selected from a group consisting of Alzheimer's disease, dementia with Lewy bodies, vascular dementia, frontotemporal lobar degeneration, epilepsy, Parkinson's disease, and a prion disease.

As used herein, the term “suffering” refers to a subject who has been diagnosed with or is predisposed to a cognitive deficit. A subject may also be referred to being “at risk of suffering” from a cognitive deficit. This subject has not yet developed characteristic pathology of the cognitive deficit, however are known to be predisposing to the cognitive deficit due to family history, being genetically predispose to developing the cognitive deficit, or diagnosed with a disease or disorder that predisposes them to developing the cognitive deficit to be treated.

As used herein, the term “nutrient” refers to any compound that nourishes a living being; more specifically, nutrients are the nutritional components in foods that an organism utilizes to survive and grow. Examples of nutrients include, but are not limited to, minerals, carbohydrates, lipids, proteins, vitamins, co-factors, inorganic salts, cations and anions typically abandoned in natural drinking water, amino acids, and organic acids.

In one preferred embodiment, DDW water having deuterium content within the range of about 90≦D/H≦135 ppm and ¹H₂¹⁶O content that is less or equal to 99.759% can be used as an ingredient of an aqueous composition comprising the compound of formula (I) where said DDW has adjuvant function for the enhancement of the effect of said compound on cognitive function. In different embodiments, the aqueous composition may be use as a nutritional composition or therapeutic composition. The composition may be used as a simple aqueous solution consisting of an amount of the compound of formula (I) dissolved in DDW having deuterium content within the range of about 90≧D/H≦135 ppm and ¹H₂¹⁶O content that is less or equal to 99.759%, or it may be formulated to comprise, additionally to said DDW and compound of formula (I), any nutrient or pharmaceutically active compound described herein that would be appropriate for different embodiments of the invention.

Choline succinate of formula (I) can be prepared by the reaction of choline hydroxide (CAS registry No. 123-41-1) with succinic acid, as it has been described in Example 1 of the international patent application PCT/RU2007/000420, international publication number WO2009/022933A1, hereby incorporated by reference.

In one embodiment, the aqueous solution contains from 0.01 to 50% of a choline salt, preferably choline succinate of formula (I); preferably, from 0.1 to 50%; more preferably, from 0.1% to 10%.

In practicing the invention, the aqueous solution of a choline salt, preferably choline succinate of formula (I), may be administered to a subject in an amount, which contains from 0.1 to 50 mg of the salt per kilogram of body weight of the subject.

In practicing the invention, the aqueous solution of a choline salt, preferably choline succinate of formula (I) may be administered to a subject in effective amounts for a period of one day or longer.

As used herein, the term “effective amount” refers to an amount of the aqueous solution of a nutrient that, when administered to a subject will provide enhancing cognitive function. The precise effective amount will vary depending on the condition and its severity and age, weight, etc., of the subject to be treated, and the mode of administration. A physician, clinician, dietary manager, or veterinarian of ordinary skill can readily determine the effective amount of said solution for enhancing cognitive function by routine experimentation.

In practicing the invention, the aqueous solution of a choline salt, preferably choline succinate of formula (I) may be administered to a subject in need thereof in amounts from 0.01 to 4 liters per subject per day.

In practicing the invention, the aqueous solution of a choline salt, preferably choline succinate of formula (I) may be used as a component of a beverage. In one embodiment, the aqueous solution contains from 0.01 to 50% of the compound of formula (I); preferably, from 0.1 to 50%; more preferably, from 0.1% to 10%.

As used herein, the term “beverage” refers to a substantially aqueous drinkable composition suitable for human consumption. In one embodiment, the beverage comprises at least 80% water by weight of the beverage.

For purposes of the invention a choline salt, preferably choline succinate of formula (I), may be prepared as the individual substance or prepared in situ during a process of preparing the beverage. When a choline salt, preferably choline succinate of formula (I), is prepared in situ, an appropriate choline base or a salt thereof and an appropriate acid or a salt thereof, preferably succinic acid or a salt thereof, are added to the beverage to form resulting choline succinate of formula (I) in situ in the beverage. For example, choline chloride and disodium succinate may be added to the beverage in the proportion of 2:1 to produce choline succinate of formula (I)) in situ in the beverage.

As used herein, the term “appropriate succinic acid salt” refers to any non-toxic succinic acid salt. Such salts include, but are not limited to, monosodium succinate, disodium succinate, potassium succinate, ammonium succinate, and mixtures and hydrates thereof.

As used herein, the term “appropriate choline salt” refers to any non-toxic choline salt. Such salts include, but are not limited to, choline hydroxide, choline chloride, choline citrate, choline bitartrate, and mixtures and hydrates thereof.

The beverage of the invention may be prepared by well-known procedures using well-known optional ingredients. Such optional ingredients generally are used individually at levels from about 0.0005% to about 10.0%, In one embodiment from about 0.005% to about 1.0% by weight of the composition. Examples of suitable optional ingredients include, but are not limited to, minerals, carbohydrates, lipids, vitamins, co-factors, buffers, flavors and sweeteners, inorganic salts, cations and anions typically abandoned in natural drinking water, taste modifying and/or masking agents, carbon dioxide, amino acids, organic acids, antioxidants, preservatives, and colorants.

Non-exclusive examples of inorganic salts typically abandoned in natural drinking water are sodium carbonate, sodium bicarbonate, potassium chloride, magnesium chloride, calcium chloride, and mixtures thereof.

Non-exclusive examples of cations are sodium, potassium, magnesium, calcium, zinc, iron, and mixtures thereof.

Non-exclusive examples of anions are fluoride, chloride, bromide, iodide, carbonate, bicarbonate, sulfate, phosphate, and mixtures thereof.

Non-exclusive examples of buffers are phosphate buffer, glycine buffer, citrate buffer, acetate buffer, carbonate buffer, tris-buffer, triethanolamine buffer, and succinate buffer.

Non-exclusive examples of flavors are synthetic flavor oils; flavoring aromatics and naturals oils such as cinnamon oil, oil of wintergreen, peppermint oils, clove oil, bay oil, anise oil, eucalyptus, thyme oil, cedar leave oil, oil of nutmeg, oil of sage, oils of citrus fruits, oil of bitter almonds, and cassia oil; plant extracts, flowers, leaves, fruits, vanilla, chocolate, mocha, coffee, apple, pear, peach, citrus such as lemon, orange, grape, lime, and grapefruit; mango, strawberry, raspberry, cherry, plum, pineapple, and apricot, and combinations thereof.

Non-exclusive examples of sweeteners are natural and synthetic sweeteners. Non-exclusive examples of natural sweeteners are naturally occurring substances, sucrose, extracts from naturally occurring substances; extracts of the plant Stevia Rebaudiana Compositae Bertoni such as stevia, steviol, rebaudiosides A-F, and dulcosides A and B; extracts of Thladiantha grosvenorii such as mogroside V and related glycosides and triterpene glycosides; phyllodulcin and its derivatives; thaumatin and its derivatives; mogrosides such as mogroside IV, mogroside V, siamenoside, and mixtures thereof; genus Siraitia including S. grosvenorii, S. siamensis, S. silomaradjae, S. sikkimensis, S. Africana, S. borneesis, and S. taiwaniana; naturally-occurring glycosides; and active compounds of plant origin having sweetening properties, and mixtures thereof. Non-exclusive examples of synthetic sweeteners are aspartame saccharin, and mixtures thereof.

Non-exclusive examples of colorants are dyes suitable for food such as those known as FD&C dyes, natural coloring agents such as grape skin extract, beet red powder, titanium dioxide, and beta-carotene, annatto, carmine, chlorophyll, paprika, and mixtures thereof.

Non-exclusive examples of organic acids are acetic acid, butyric acid, succinic acid, fumaric acid, malic acid, pyruvic acid, glutamic acid, citric acid, omega-3 unsaturated acids, linoleic acid, linolenic acid, eicosapentaenoic acid, docosahexaenoic acid, aspartic acid, and mixtures thereof.

Non-exclusive examples of amino acids are L-Tryptophan, L-Lysine, Methionine, Threonine, Levocarnitine, and L-carnitine.

Non-exclusive examples of vitamins are thiamin, riboflavin, nicotinic acid, panthothenic acid, biotin, folic acid, pyridoxine, vitamin B12, lipoic acid, vitamin A, vitamin D, vitamin E, ascorbic acid, choline, carnitine; alpha, beta, and gamma carotenes; vitamin K, and mixtures thereof.

Non-exclusive examples of co-factors are thiamine pyrophosphates, flavin mononucleotide, flavin adenine dinucleotide, nicotinamide adenine dinucleotide, pyridoxal phosphate, biotin, tetrahydrofolic acid, Coenzyme A, coenzyme B12, lipoyllysine, nicotinamide adenine dinucleotide phosphate, 11-cis-retinal, 1,25-dihydroxycholecalciferol and mixtures thereof.

Non-exclusive examples of antioxidants are Vitamin E, ascorbic acid, carotenoids, aminoindoles, Vitamin A, uric acid, flavonoids, polyphenols, herbal antioxidants, melatonin, lipoic acids, and mixtures thereof.

In one embodiment, the aqueous solution of a choline salt, preferably a choline succinate of formula (I), may be used as a component of a dietary supplement. In one embodiment, the aqueous solution contains from 0.01 to 50% of the compound of formula (I); preferably, from 0.1 to 50%; more preferably, from 0.1% to 10%.

As used herein, the term “dietary supplement” refers to a product taken by mouth that contains a dietary ingredient intended to supplement the diet.

In one embodiment, the aqueous solution of a choline salt, preferably choline succinate of formula (I), may be used as a component of a medical food.

In one preferred embodiment, the aqueous solution contains from 0.01 to 50% of choline succinate of formula (I); preferably, from 0.1 to 50%; more preferably, from 0.1% to 10%.

As used herein, the term “medical food” refers to a food which is formulated to be consumed or administered enterally under the supervision of a physician and which is intended for the specific dietary management of a disease, condition, or disorder.

In one embodiment, the aqueous solution of the aqueous solution of a choline salt, preferably choline succinate of formula (I), may be used as a component of a pharmaceutical composition. In one preferred embodiment, the aqueous solution contains from 0.01 to 50% of a choline salt, preferably choline succinate of formula (I) preferably, from 0.1 to 50%; more preferably, from 0.1% to 10%.

As used herein, the term “pharmaceutical composition” refers to any composition comprising at least one pharmaceutically active ingredient and at least one other ingredient, e.g. diluent, excipient, or carrier.

In one embodiment, water having deuterium content from about 90 to about 135 ppm and ¹H₂¹⁶O isotopologue up to 99.759% is used as an adjuvant component of a pharmaceutical composition at levels from 0.0001 to 99.999999% by weight of the composition, said composition comprising the compound of formula (I) (interchangeably termed herein as “pharmaceutical composition of the invention of “the pharmaceutical composition”).

Pharmaceutical compositions of the invention may comprise optional ingredients. Such optional ingredients generally are used individually at levels from about 0.0005% to about 10.0%, preferably from about 0.005% to about 1.0% by weight of the composition. Nonexclusive examples of optional ingredients include absorbents, buffering agents, colorants, solvents and co-solvents, coating agents, direct compression excipients, lubricants, sweetening agents, anti-fungal preservatives, antimicrobial preservatives, clarifying agents, emulsifying agents, antioxidants, surfactants, tonicity agents, and viscosity increasing agents.

In practicing the invention, the pharmaceutical composition may be prepared in a wide variety of different dosage forms including, but are not limited to, solutions, spray, aerosols, elixirs, syrups, gels, and capsules.

In practicing the invention, the pharmaceutical composition may be administered to a subject in need thereof by different routes including, but are not limited to, topical, oral, sublingual, parenteral (e.g. intravenous, subcutaneous, or intramuscular injections), nasal, rectal, vaginal, and percutaneous administration.

In practicing the invention, a pharmaceutical composition of the invention may be used in a combination with a cognitive enhancer or a mixture thereof.

As used herein, the term “cognitive enhancer” refers to any agent useful for enhancing cognitive function in a subject in need thereof.

The cognitive enhancers include, but are not limited to, agents interacting with receptors, agents interacting with enzymes, agents interacting with cytokines, agents interacting with gene expression, agents interacting with heat shock proteins, agents interacting with hormones, agents interacting with Ion channels, agents interacting with nerve growth factors, agents interacting with re-uptake transporters (psychostimulants), agents interacting with transcription factors, antioxidants, metal chelators, natural Products, nootropics (“agents without mechanism”), peptides; agents preventing amyloid-beta aggregation, e.g. ligands interacting with amyloid-beta, inhibitors of serum amyloid P component binding, vaccines against amyloid-beta, antibodies against amyloid-beta, agents interacting with tau; small molecules preventing tau aggregation, e.g. ligands interacting with tau, vaccines against tau, antibodies against tau; stem cells, and miscellaneous.

As used herein, the term “agent” refers to any substance, molecule, compound, methodology and/or biologic agent for use in the prevention, treatment, management and/or diagnosis of a disease or disorder.

Agents interacting with receptors include, but are not limited to, agents interacting with acetylcholine receptors, e.g. muscarinic acetylcholine receptors M1, M2, M3, M4, and M5 (mAChRs) and nicotinic acetylcholine receptors alpha4beta2, alpha3beta4 and alpha7; agents interacting with adenosine receptors; agents interacting with adrenergic receptors; agents interacting with angiotensin receptors; agents interacting with cannabinoid receptors; agents interacting with chemokine receptors; agents interacting with dopamine receptors; agents interacting with endothelin receptors; agents interacting with estrogen receptors; agents interacting with GABA receptors; agents interacting with galanin receptors; agents interacting with glutamate receptors; agents interacting with glutamate receptors; agents interacting with glutamate receptors; agents interacting with G-protein coupled orphan receptors; agents interacting with histamine receptors; agents interacting with insulin receptors; agents interacting with liver X receptors; agents interacting with neurotensin receptors; agents interacting with nociceptin (ORL1) receptors; agents interacting with peripheral benzodiazepine receptors; agents interacting with peroxisome proliferator-activated (PPARs) receptors; agents interacting with prostaglandin receptors; agents interacting with purinergic receptors; agents interacting with receptor for advanced glycation end products (RAGE); agents interacting with Retinoid X receptor; agents interacting with Ryanodine receptor; agents interacting with serotonin receptors, e.g. 5-HT1A, 5-HT2, 5-HT3, 5-HT4, and 5-HT6; agents interacting with sigma receptor; agents interacting with somatostatin receptor; agents interacting with sphingosine-1-phosphate receptor; agents interacting with tachykinin receptor; agents interacting with tumor necrosis factor receptor 1; and agents interacting with any receptor to be discovered and developed to enhance cognitive function. Nonexclusive examples of agents interacting with muscarinic acetylcholine receptors M1 include orthosteric mACh M1 receptor agonists, allosteric mACh M1 receptor agonists, vincamine-type compounds, cevimeline, xanomeline, milameline, alvameline, talsaclidine, AF-267B (NGX-267), WAY-132983, sabcomeline, MCD-386, CDD-0102, azpet, AC-42, AC-260584, AC-262271, GSK-1034702, AE510about 90, BQCA, TBPB, AM-831, N-desmethylclozapine, vincamine, ML169, RGH-10885; and any agent to be discovered or developed in future. Nonexclusive examples of agents interacting with muscarinic acetylcholine receptors M2 include BIBN-99 and SCH-217443. Nonexclusive examples of agents interacting with nicotinic acetylcholine receptors include alpha4beta2 and alpha3beta4 nicotinic acetylcholine receptor agonists such as varenicline, pozanicline, Ispronicline, AZD-1446, Lobeline, Sofinicline, ABT-560, NS-9283, S-35836-1, SAZETIDINE-A, SR-16584, SUVN-F91201, SUVN-911. Further, nonexclusive examples of agents interacting with nicotinic acetylcholine receptors include alpha7 nicotinic acetylcholine receptor agonists such as ABT-126, EVP-6124, GTS-21, TC-5619, ABT-272, A-867744, APL-1, AZT-2, BMS-about 902483, BNC-1881, JNJ-1930942, PheTQS, S-24795, SEN-34625/WYE-103914, SEN-15924/WAY-361789, SKL-A4R, UCI-40083,; and any agent to be discovered or developed in future. Nonexclusive examples of agents interacting with adenosine receptors include, but are not limited to, caffeine, tozadenant, and any agent to be discovered or developed in future. Nonexclusive examples of agents interacting with adrenergic receptors include, but are not limited to, buflomedil, ORM-12741, carvedilol, and any agent to be discovered or developed in future. Nonexclusive examples of agents interacting with angiotensin receptors include, but are not limited to, tozadenant, losartan, candesartan, telmisartan, valsartan, eprosartan, irbesartan, and any agent to be discovered or developed in future. Nonexclusive examples of agents interacting with cannabinoid receptors include, but are not limited to, Dronabinol, Rimonabant, and any agent to be discovered or developed in future. Nonexclusive examples of agents interacting with chemokine receptors include, but are not limited to, RAP-310 and any agent to be discovered or developed in future. Nonexclusive examples of agents interacting with dopamine receptors include, but are not limited to, dexpramipexole, seridopidine, pridopidine, PF-03800130, and any agent to be discovered or developed in future. Nonexclusive examples of agents interacting with endothelin receptors include, but are not limited to, ENDG-6010 and any agent to be discovered or developed in future. Nonexclusive examples of agents interacting with estrogen receptors include, but are not limited to, phyto-beta-SERM, liquiritigenin, (−)-epigallocatechin-3-gallate, dehydroepiandrosterone, and any agent to be discovered or developed in future. Nonexclusive examples of agents interacting with GABA receptors include, but are not limited to, RG-1662, AC-4402, C-21191, RO4938581, UC-1011, CGP36742, CGP36742, CGP51176, CGP56433, CGP63360, (R or S)-ACBPA, HT-2157, and any agent to be discovered or developed in future. Nonexclusive examples of agents interacting with galanin receptors include, but are not limited to, HT-2157 and any agent to be discovered or developed in future. Nonexclusive examples of agents interacting with glutamate receptors include, but are not limited to, agents interacting with AMPA receptors such as Piracetam, Oxiracetam, Dimiracetam, NT-24336, Nefiracetam, AMPAkines, CX-516, CX-717, CX-1739, S-47445, CX-546, CX-554, Farampator, biarylpropylsulfonamides, PF-04958242, LY-392098, LY-404187, LY-503430, mibampator, compound 17i, compound 9a, PF-04778574, (R,R)-PIMSD, benzothiadiazides, Cyclothiazide, IDRA-21, S-18986, 1-hydroxyazoles, and any agent to be discovered or developed in future. Nonexclusive examples of agents interacting with glutamate receptors include, but are not limited to, agents interacting with NMDA receptors such as Memantine, Neramexane, EVT-103, Mnemosyne, NRX-1059, and any agent to be discovered or developed in future. Nonexclusive examples of agents interacting with glutamate receptors include, but are not limited to, agents interacting with metabotropic glutamate receptors (mGluRs) such as ADX-71149, RG-1578, RG-70about 90, BCI-838, BCI-632, STX-107, ADX-63365, DT-2228, RO-4491533, VU-0430644, and any agent to be discovered or developed in future. Nonexclusive examples of agents interacting with G-protein coupled orphan receptors include, but are not limited to, ESN-502, GPR3, GPR12, GPR27, GPR31, GPR52, GPR78, GPR-83, GPR135, GPR139, GPR151, GPR153, and GPR173 antagonists, Mas-related G-protein coupled receptor antagonists, RGS-14, and any agent to be discovered or developed in future. Nonexclusive examples of agents interacting with histamine receptors include, but are not limited to, ABT-288, AZD-5213, JNJ-17216498, S-38093, SAR-110894, Irdabisant, MK-3134, PD-9475, Ciproxifan, EVT-501, JNJ-10181457, SUVN-G1031, and any agent to be discovered or developed in future. Nonexclusive examples of agents interacting with insulin receptors include, but are not limited to, intranasal insulin, AGT-160, SYN-200about 90510RU, and any agent to be discovered or developed in future. Nonexclusive examples of agents interacting with liver X receptors include, but are not limited to, GW3965, T0about 901317, and any agent to be discovered or developed in future. Nonexclusive examples of agents interacting with neurotensin receptors include, but are not limited to, NT-69-L and any agent to be discovered or developed in future. Nonexclusive examples of agents interacting with nociceptin (ORL1) receptors include, but are not limited to, PF-454583, PF-4926965, and any agent to be discovered or developed in future. Nonexclusive examples of agents interacting with peripheral benzodiazepine receptors include, but are not limited to, SSR-180575, BAY-85-8102, and any agent to be discovered or developed in future. Nonexclusive examples of agents interacting with peroxisome proliferator-activated (PPARs) receptors include, but are not limited to, Rosiglitazone, Pioglitazone, Mitoglitazone, DSP-8658, G-15750, and any agent to be discovered or developed in future. Nonexclusive examples of agents interacting with prostaglandin receptors include, but are not limited to, EP2 antagonists, TG-6-10-1, and any agent to be discovered or developed in future. Nonexclusive examples of agents interacting with purinergic receptors include, but are not limited to, P2X7R antagonists, P2X7 antagonists, and any agent to be discovered or developed in future. Nonexclusive examples of agents interacting with receptor for advanced glycation end products (RAGE) include, but are not limited to, TTP-4000, DBT-066, FPS2-BM, FPS-ZM1, A-992401, humanized anti-RAGE antibody, PF-04494700, and any agent to be discovered or developed in future. Nonexclusive examples of agents interacting with Retinoid X receptor include, but are not limited to, Bexarotene, Tamibarotene, and any agent to be discovered or developed in future. Nonexclusive examples of agents interacting with Ryanodine receptor include, but are not limited to, Dantrolene and any agent to be discovered or developed in future. Nonexclusive examples of agents interacting with serotonin receptors 5-HT1A, 5-HT2, 5-HT3, 5-HT4, and 5-HT6 include, but are not limited to, F-15599, Asenapine, PRX-3140, Velusetrag, RQ-00000009, SUVN-D1003019, SUVN-1004028, Latrepirdine, Lu-AE58054, SB-742457, AVN-101, AVN-211, AVN-322, ABT-354, SAM-760, SUVN-502, SUVN-507, SYN-114, SYN-120, Pyrazolo[1,5-a]pyrimidines, 5-HT6 receptor antagonists, and any agent to be discovered or developed in future. Nonexclusive examples of agents interacting with sigma receptor include, but are not limited to, Fluvoxamine, Cutamesine, Anavex-2-73, Anavex-1-41, MC-113, (±)-PPCC, (−)-MR22, and any agent to be discovered or developed in future. Nonexclusive examples of agents interacting with somatostatin receptor include, but are not limited to, NNC-26-9100 and any agent to be discovered or developed in future. Nonexclusive examples of agents interacting with sphingosine-1-phosphate receptor include, but are not limited to, modulators of the receptor such ABT-363 and any agent to be discovered or developed in future. Nonexclusive examples of agents interacting with tachykinin receptor include, but are not limited to, SSR-241586 and any agent to be discovered or developed in future. Nonexclusive examples of agents interacting with tumor necrosis factor receptor 1 include, but are not limited to, negative allosteric modulators of the receptor such as PD-2015, PD-2016, and any agent to be discovered or developed in future.

Agents interacting with enzymes include, but are not limited to, agents interacting with Acetylcholinesterase (AChE) and Butyrylcholinesterase (BChE); agents inhibiting AChE and other biological targets; dual AChE inhibitors and AChE receptor ligands; dual AChE and amyloid-beta inhibitors; dual AChE inhibitors and antioxidants; dual AChE and beta-secretase-1 or gamma-secretase inhibitors; dual AChE inhibitors and calcium channel blockers; dual AChE inhibitors and cannabinoid receptor antagonists; dual AChE and fatty acid amide hydrolase inhibitors; dual AChE inhibitors and histamine H3 receptor antagonists; dual AChE and monoamine oxidase inhibitors; dual AChE inhibitors and metal chelators; dual AChE inhibitors and N-methyl-D-aspartic acid receptor channel blockers; dual AChE inhibitors and platelet activating factor antagonists; dual AChE and serotonin transporter inhibitors; dual AChE and sigma receptor inhibitors; agents interacting with alpha-Secretase; agents interacting with beta-Secretase; agents interacting with gamma-Secretase; gamma-Secretase inhibitors; gamma-Secretase modulators; inhibitors of gamma-Secretase activating protein; Notch pathway inhibitors; agents interacting with beta-Hexosam inidase; agents interacting with 11beta-Hydroxysteroid Dehydrogenase; agents interacting with Calpain; agents interacting with Carbonic Anhydrase; agents interacting with Caspases; agents interacting with Catechol-O-methyltransferase; agents interacting with Cathepsin; agents interacting with Cholesterol 24Shydroxylase (CYP46A1), agents interacting with Cyclooxygenase; agents interacting with D-Amino Acid Oxidase; agents interacting with Glutaminyl Cyclase; agents interacting with Glyceraldehyde-3-Phosphate Dehydrogenase; agents interacting with Glycogen Synthase Kinase-3; agents interacting with Guanylyl Cyclase; agents interacting with Heme Oxygenase; agents interacting with Histone Deacetylase; agents interacting with HMG-CoA Reductase; agents interacting with Insulin-degrading Enzyme; agents interacting with GSK-3beta; agents interacting with PKC; agents interacting with Kynurenine MonoOxygenase and Kynurenine Transaminase II; agents interacting with 5-Lipoxygenase; agents interacting with Monoamine Oxidase; agents interacting with Peptidyl-prolyl cis-trans Isomerase D; agents interacting with Phosphodiesterases; agents interacting with Phospholipases A2 and D2; agents interacting with Plasminogen Activator; agents interacting with Poly ADP-Ribose Polymerase; agents interacting with Prolyl Endo Peptidase; agents interacting with Prostaglandin D and E Synthases; agents interacting with Protein Kinase C; agents interacting with Protein Tyrosine Phosphatase; agents interacting with Rac1 GTPase; agents interacting with Ras Farnesyl Transferase; agents interacting with S-Adenosylhomocysteine Hydrolase; agents interacting with Sirtuin; agents interacting with Steroid sulfatase; agents interacting with Transglutaminase; agents interacting with Ubiquitin Carboxylterminal Hydroxylase; and agents modulating O-linked N-Acetylglucosaminidase. Nonexclusive examples of agents interacting with enzymes include Tacrine, Donepezil, Donepezil+memantine; Rivastigmine; NAL-8822; Galantamine, NAL-8801, Memogain, Huperzine A, XEL-001 HP, WIN-026, Shen Er Yang, Methanesulfonyl fluoride, Bisnorcymserine, Bis-(7)-tacrine, FS-0311, Huprines, NP-0336, SPH-1285, Caproctamine, MHP-133, Ro-46-5934, NP-61, IDN-5706, IQM-622, Bisnorcymserine, Lipocrine, Memoquin, Coumarin derivatives, tacrine-chromene derivatives, ITH-4012, ITH-12118, Compound 20, MIQ-001, AMR-109, FUB833, Ladostigil, HLA20A, PMS-777, PMS-1339, RS-1259, SP-04, Etazolate, Bryostatin-1; NP-17, NP-21, NPM-01, NPM-05B1, and NPM-05B2; LY-2886721, CTS-21166, E-2609, HPP-854, MK-8931, RG-7129, AMG-0683, AZ-13, 7-aza-indole compounds; hydroxyethylamine beta-secretase-1 inhibitors; JNJ-715754; KMI-008, KM1-358, KMI-370, KMI-420, KMI-429, KMI-574, KMI-1027, and KMI-1303; L-655240, TAK-070, WAY-258131, antibodies directed against the beta-secretase cleavage site of AbetaPP; brain-targeted BACE1 antibody; Avagacestat, NIC5-15, E-2212, (−)-GSI-1; MRK-560, NGP-555, RO-02, Tarenflurbil, CHF-5074, EVP-0962, AZ-4800, BIIB-042, GSM-2, GSM-10h; SPI-014, SPI-1802, SPI-1810, and SPI-1865; IC-200155, AGT-0031, ABT-384, KR-1-2; UE-1961, UE-2811, and UE-2343; A-705253, ABT-957, AK-295, SNJ-1945, NWL-117, Tolcapone, Cerecor, Aloxistatin; Acetyl-L-leucyl-L-valyl-L-lysinal; AAV-CYP46A1, AS-2651816-00, PBD-150, PQ-912, Omigapil, Tideglusib, AX-9839; CG-9, CG-701338, CG-701446, and CG-701448; CP-70949; Dual GSK-3·/c□sein kin□se 2 modul□tors; GSK-3beta·inhibitors; JI-7263, NNI-362, NP-101020, SN-2127, TWS-119, VP1.15; DM-204; sGC-1016; GT-715 and GT-1061; OB-28, EVP-0334, 4-Phenylbutyrate; CHDI-3about 90576 and CHDI-00381817; Crebinostat; KAR-3010, KAR-3084, and KAR-3166; LB-201 and LB-205; Pitavastatin, Atorvastatin, Pravastatin, Simvastatin, NST-0037, acetyl-L-carnitine, Masitinib; AIK-2, AIK-2a, AIK-2c, and AIK-21; Beta carboline alkaloids; Cadeprin, Casein kinase 1δ inhibitors; CDK5/p25 inhibitors; CZC-25146; Dasatinib; DYRK-1 alpha protein kinase inhibitors; DYRK-1 alpha protein kinase inhibitors; FRAX-120, FRAX-355, and FRAX-486; G-2019S, Hydroxy-fasudil; KIBRA pathway modulators; LDN-22684; LRRK2 inhibitors; LRRK2 inhibitors; microtubule affinity regulating kinase inhibitors; microtubule affinity regulating kinase 3 inhibitors; Minokine, NNI-351, P-005, SEL-103, SEL-141, Sorafenib, TTT-3002, URMC-099-C; protein kinase c-raft inhibitors; CHDI-003940246 and CHDI-00340246; PF-04859989; Minocycline, Ladostigil, RG-1577; OG-45, VAR-10200, VAR-10300, Rasagiline, Safinamide, O-GIcNAcase modulators; GIcNAcstatin, NButGT, SEG-4, Thiamet-G, Etazolate, PF-02545920, Lu-AF11167, AMG-7980, AVE-8112, BCA-about 909, Cilostazol, Dual PDE10/PDE 2 inhibitors; GEBR-7b, ITI-002A, 1C-200214, and ITI-214; OMS-182410, PDE2A inhibitors; PDE7 inhibitors; PDE9 inhibitors; PDE10inhibitors; PF-999; Sildenafil and Tadalafil; THPP-1; Rilapladib; Icosapent ethyl; IMD-4482; E-7016; MP-124; AL-309; rosmarinic acid; HF-0220; AAD-2004; APH-0703; Bryostatin-1; DCP-LA; PKC epsilon activators; LDN-33960; Cytotoxic Necrotizing Factor 1; Sanquinarinium chloride; LNK-754; L-002259713; Resveratrol; Selisistat; INDUS-815C; DU-14; CHDI-00339864 and CHDI-00316226; Usp14 inhibitors; and any agents interacting with enzymes to be discovered and developed in future.

Agents interacting with cytokines include, but are not limited to, agents inhibiting microglia activation and production of pro-inflammatory cytokine (e.g. TNF-alpha, IL-1beta, IL-6, and IL-8). Nonexclusive examples of agents interacting with cytokines include TT-301; TT-302; Minozac; AD-16; SEN-1176; Infliximab; an antibody against the interleukin-12 subunit p40; and any agents interacting with cytokines to be discovered and developed in future.

Agents interacting with gene expression include, but are not limited to, Beperminogene perplasmid; RVX-208; AAV-CYP46A1; AZ-AAV9; HSD17B10; PRO-289; SynCav; Inventiva; and any agents interacting with gene expression to be discovered and developed in future.

Agents interacting with heat shock protein include, but are not limited to, heat shock protein about 90 inhibitors PU-H71, PU-3, PU24FCI, PU-DZ8, E102, KU-32; and any agents interacting with heat shock protein to be discovered and developed in future.

Agents interacting with hormones include, but are not limited to, thyrotropin-releasing hormone and its analogues and derivatives thereof such as Taltirelin and KPS-0373; gonadotropin-releasing hormone agonist such as Leuprolide acetate implant; growth hormone releasing factor derivative such as Tesamorelin; and any agents interacting with hormones to be discovered and developed in future.

Agents interacting with ion channels (non-receptors) include, but are not limited to, blockers of L-type voltage-gated calcium channels such as ARC-029, nilvadipine, and Isradipine; ARC-031 and ARC-031-SR; RNS-60; blockers of T-type voltage-gated calcium channels such as ZSET-1446; sodium channel blockers such as AD-NO2; and any agents interacting with ion channels (non-receptors) to be discovered and developed in future.

Agents interacting with nerve growth factor include, but are not limited to, NeuroAid; NeuroAiD II; CERE-110; GM-607; MIM-D3; PYM-50028; T-817MA; NsG-0202; AL-209, AL-309; MRS-001; Catecholamine derivatives; CB-1, CB-2, and CB-3; 7,8-Dihydroxy-flavone; FC29 peptide; Gambogic amine and gambogic amide; Gedunin; JRP-655; 4-methylcatechol; ND-602; and any agents interacting with nerve growth factor to be discovered and developed in future.

Agents interacting with re-uptake transporters (psychostimulants) include, but are not limited to, re-uptake inhibitors of the monoamine transporters such as Methylphenidate, Dexmethylphenidate, Modafinil, Armodafinil, Atomoxetine, Lisdexamfetamine, Indeloxazine, NS-2359, Lu-AA42202; agent interacting with creatine transporter AM-285; glycine transporter-1 inhibitors such as AS-1522489-00 and RO-4543338; dopamine transporter inhibitors such as PD-2005 and MLR-1017; selective serotonin transporter inhibitors such as Thiethylperazine, Fluoxetine, and Citalopram; and any agents interacting with re-uptake transporters (psychostimulants) to be discovered and developed in future.

Agents interacting with transcription factors include, but are not limited to, modulators of cAMP response element-binding (CREB) protein; inhibitors of exchange protein directly activated by cAMP (EPAC); and any agents interacting with transcription factor to be discovered and developed in future.

Antioxidants include, but are not limited to, acetyl-L-carnitine, curcumin, Gingko biloba extracts such as EGb 716, (R)-α-lipoic acid, melatonin, morin, trolox, vitamin C, and vitamin E, edaravone, idebenone, tirilazad, MitoQ, MitoVitE, MitoPBN, MTP-131, VP-20629; manganoporphyrine antioxidants such as AEOL-10113, AEOL-10150, AEOL-10201 and AEOL-11207; CNB-001; DL-3-n-butylphtalide; FRP-0924; IAC; Lipid soluble antioxidants; Lipocrine and Memoquin dual Acetylcholinesterase inhibitors and antioxidants; NPS-0155; PAN-811; S-52; peoniflorin; 2,2′-pyridoin; quetiapine; stemazole; zeatin; dual free radical scavengers and Abeta· binding lig□nds; □nd □ny □nitioxid□nt to be discovered □nd developed in future.

Metal chelators include, but are not limited to, calcium and zinc chelators such as DP-b99, Aom-0937, PBT-2, DP-460, AEN-100; iron chelators such as Deferoxamine; non-selective metal chelators such as HLA20A, PBT-3, PBT-4; copper chelator PA-1637; dual iron-chelating agent and MAO-B inhibitors such as VAR-10200, VAR-10300; and any metal chelator to be discovered and developed in future.

Natural products include, but are not limited to, herbal extracts such as Mentat, SK-PC-B70M, KD-501, YY-280, prenylflavanone compounds PPLs, PTX-200; melatonin formulations such as melatonin and Circadin; Resveratrol; RPh-201; VR-040; Exebryl-1; Taisi; Alpha-mangostin; Anatabine; Andrographis paniculata leaves extract; Apomorphine; AX-00111; Axona; Bacopa monnieri; Baicalein; Beta-asarone; BT-11; BV-7003; Cabernet Sauvignon; Carvacrol; Catechins; Celastrol; Celastrus paniculatus; Chelerythrine; Cinnamon extract; Coumarins; Cryptotanshinone; phenolic compounds; Curcumin; DL-3-n-butylphtalide; DX-9386; Ecdysterones; (−)-Epigallocatechin-3-gallate; ESP-102; Eucommia ulmoides Oliv. Bark; Flavonoids; Fortasyn Connect; Fulvic acid; Gallic acid; Garlic extract; Gastrodin; Ginger root extracts; Gingko biloba; Grape-derived polyphenolics; Guanosine; Hederacolchidide-E; Heme; Hopeahainol A; HSH-971; HX-106; Hyperoside; IB-10C179; Icariin; IDN-5706; Kaempferol; Loganin; Luteolin; Medical Food Cocktail; Menthol; Morin; Myrcetin; Naringin; Nordihydroguaiaretic acid; O4; Obovatol; Oleuropein; Oren-gedoku-to; OroxylinA; 1,2,3,4,6-penta-O-galloyl-beta-D-glucopyranose; Piceatannol; Pinocembrin; Polyphenol derivatives; Procyanidins; Prosopis cineraria; Puerarin; Pycnogenol; Quercetin; Rosmarinic acid; Rutin; Saffron; Salidroside; S-allyl-L-cysteine; Silibinin; Sinapic acid; Souvenaid; Substance P the tachykinin undecapeptide; Syringin; Tannic acid; alpha-Tocopherol quinone; Thymol; Total coptis alkaloids; Ursolic acid; Vitamin A; Vitamin B12; Vitamin D; Withania somnifera; Wuzi Yanzong Granule; Yokukansan; Zokumei-to; bacosides A and B; Vitamin B15 (or pangamic acid), glycine, N,N-dimethyl glycine; D-cycloserine; omega-3 polyunsaturated fatty acids such as Hexadecatrienoic acid (HTA), α-Linolenic acid (ALA), Stearidonic acid (SDA), Eicosatrienoic acid (ETE), Eicosatetraenoic acid (ETA), Eicosapentaenoic acid (EPA), Heneicosapentaenoic acid (HPA), Docosapentaenoic acid (DPA), Clupanodonic acid, Docosahexaenoic acid (DHA), Tetracosapentaenoic acid, and Tetracosahexaenoic acid; and any natural product to be discovered and developed in future.

Nootrpics include, but are not limited to, LSL-001; N-251; PF-03049423; TPM-189; VI-1121; ASP-0777; RO-5508887; SEP-363856; TAK-357; AC-0523; AFX-929; AVN-457,AVN-458, andAVN-492; CPC-001; CWF-0804; D-130; D-180; GSK-2647544; J-147; JAY2-22-33; JWB1-84-1; KD-about 901; KU-046; LNK-3186 and LNK-3248; Maltoyl p-coumarate; MeN061016-1; MPP-26; NNZ-2591; NXD-about 9062; NXT-182; OG-635; Pentylenetetrazole; PNB-03, PNB-04, and PNB-05; PTI-125; RP-4000; SEL-103; SKL-A4R; TYP-1; and any nootropic to be discovered and developed in future.

Peptides include, but are not limited to, Cerebrolysin; Cortexin; Davunetide; AM-111; Etanercept; FGL; Glypromate; NNZ-2566; AL-408; Alzimag; C3bot peptides; COG-112, COG-133, and COG-1410; G-79; KIBRA pathway modulators; Leptin; MT-007; Netrin-1; NNZ-4921 and NNZ-4945; NRG-101; NT-1 and NT-2; NX-210; Pepticlere; PP-0301; RAP-310; RG-01, RG-09, and RG-018; SX-AZD1; XD4; Colostrinin; and any peptide to be discovered and developed in future.

Agents preventing Amyloid-beta aggregation include, but are not limited to, Tafamidis, Eprodisate, ARC-029, Davunetide, APH-0703, Doxycycline hyclate, ELND-005, SOM-0226, AAD-2004, Beta amyloid modulators; BLU-8499; DWP-0about 9031; Exebryl-1; NP-61; Systebryl; Amyloid-beta oligomer cellular prion protein binding inhibitors; peptidomimetic compounds that interfere with Amyloid-beta aggregates; Amyloid-beta/tau protein aggregation inhibitors; Amyloid-derived diffusible ligands; ARN-4261 and ARN-2966; AVCRI-104P4; AVN-457,AVN-458 andAVN-492; AZP-2006; Beta-amyloid/alpha-synuclein/tau aggregation inhibitors; Beta amyloid beta sheet formation inhibitors; Beta-amyloid modulators targeting Amyloid-beta oligomers and misfolded proteins; Beta-amyloid precursor protein modulators; BMS-869780; BTA-EG4; C36; Caprospinol; Carvedilol; CLR01; CLR-097; Cotinine; Daunomycin; DBT-1339; Enoxaparin; glycosaminoglycan/carbohydrate compounds; Galantamine; haw-AD-14; HO-4160; Imipramine; IPS-04001, IPS-04001 and IPS-04003; KMS-88; Minocycline; NPT-4003; Rifampicin; Rolitetracycline; SD-1002; SEN-1500; SP-08; TAK-070; Tetracycline(s); VP-20629; AGT-160; EDN-OL1; NPT-001; compounds capable of blocking the transformation of human spherons into plaques; inhibitors of serum amyloid P component binding to amyloid fibrils such as Ro-63-8695; vaccines against amyloid-beta such as AN-1792, Affitope AD-02, CAD-106, Vanutide cridificar, ACI-24, Affitope-AD-03, UB-311, V-950, ABvac40 and ABvac42, ADepVac, ALZ-101, ALZ-301, vaccine that activates antibodies against Amyloid-beta protein, Amyloid-beta 3-10 DNA vaccination, Ankyrin G, BAN-2203, BBS-1 BACE inhibitor mAb vaccine, C12, EB-101, Glatimer acetate, MER-5101, Mimovax, NU-700, Recombinant adenovirus vector vaccine, RV-03, SeV-amyloid beta RNA vaccine; Antibodies against amyloid-beta such as Bapineuzumab, Solanezumab, Gantenerumab, Crenezumab, GSK-933776A, intravenous immunoglobulins, Immune globulin intravenous human, Octagam, Flebogamma DIF 10% (or 5%), BAN-2401, NI-101, PF-05236812, RN6G, SAR-228810, 4E10, 6F6 (GSK), 9D5, A-887755, A-992401, Ab40-4-42, ACU-193, AD-0802, AGT-160, ALZ-201, Amyloid precursor protein C-terminal fragment-targeted monoclonal antibodies, Anti-amyloid beta antibodies, Brain-targeted BACE1 antibody, CPHPC+antibody, DLX-212, Fully human monoclonal antibodies, IN-N01, IN-N01-OX2, Lpathomab, MDT-2007, naturally occurring single chain antibodies of Camelidae, NEOD-001, STC-103; and any agents preventing Amyloid-beta aggregation to be discovered and developed in future.

Ligands interacting with Tau include, but are not limited to, small molecules preventing tau aggregation such as methylene blue (TRx-0014), LMT-X, PBT-2, Tideglusib, BMS-241027, PP2A stimulators, Amyloid-beta/tau protein aggregation inhibitors, Astemizole and Lansoprazole, Berberine, Bezafibrate, BLV-0703, EpothiloneD, Fulvic acid, Insulin intranasal, L-3-n-butylphthalide, NBB BSc3504, NC-11813, NP-111001 derivatives, NPT-002, Protein phosphatase methylesterase 1 (PME1) inhibitors, Protein phosphatase 2A stimulators, tau detoxifying compounds targeting tau-mediated cytotoxicity (ReS3-T, ReS8-T, ReS10-T and ReS19-T), ReS9-S7 and ReS12-S, SIG-1012 and SIG-1106, small molecule Tau protein modulators, Sodium selenite, Tau oligomer inhibitors, Tau phosphorylation inhibitors, agents targeting tau kinase, Thiamet-G, THQ-4S and THQ-55, TRx-0237, Tubastatin A; ligands interacting with Tau such as T-777, T-807, and 1-808; Vaccines against Tau such as Recombinant misfolded truncated tau protein vaccine, RV-03; Antibodies against tau and a-synuclein such as humanized tau monoclonal antibodies, NI-105, PD-0805, T01-OX2, TauC3 monoclonal antibody, Tau protein modulators, monoclonal antibody targeted to tau, TOC-1; and any ligands interacting with Tau to be discovered and developed in future.

Stem cells include, but are not limited to, GDNF/BDNF-producing glial and brain-derived stem cells, human neural stem cell, mesenchymal bone marrow-derived stem cell, NSI-189, NSI-566RSC, Neurostem-AD, adult mesenchymal precursor stem cells, allogenic umbilical cord stem cells, brain-derived stem cells, cord blood stem cells (e.g. CPG23NEUR), glial progenitor cells, human neural progenitor cells, human umbilical cord blood cells, stem cell stimulators (e.g. NBI-18), NeurotrophinCell, NGN-about 9079, ReN-004, ReN-005, compounds inducing stem cell differentiation (e.g. SP-sc4 and SP-sc7), Allopregnanolone, Granulocyte colony-stimulating factor, neurogenesis inductors (e.g. Valproic acid); and any stem cells to be discovered and developed in future.

Miscellaneous cognitive enhancers include, but are not limited to, autophagy inducers (e.g. JRP-about 900), cellular homeostasis modulators (e.g. CNS-102), glycan inhibitors, macrophage migration inhibitory factor (MIF) inhibitors (e.g. INV-88), MicroRNA (miRNA) mimetics, Proteasome-gating modulators, Synaptic vesicle glycoprotein 2A (SV2A) ligand levetiracetam, Low-dose therapy Levetiracetam, Brivaracetam; anti-inflammatory agents such as dapsone, indomethacin, nonsteroidal anti-inflammatory drugs (NSAIDs) and COX-2 inhibitors; micronutrients such as selenium and zinc; water having deuterium content from 0 to 89 ppm; water having 17-oxygen content from 0 to 500 ppm; water having 18-oxygen content from 0 to 3000 ppm; and any cognitive enhancer to be discovered and developed in future.

For enhancing cognitive function, cognitive enhancers can be used as a mixture thereof. Nonexclusive examples of such mixtures include combinations of cognitive enhancer with water selected from the group consisting of water having deuterium content from 0 to 89 ppm, water having 17-oxygen content from 0 to 500 ppm, and water having 18-oxygen content from 0 to 3000 ppm.

In practicing the invention, an effective amount of the aqueous solution of invention may be encapsulated into a digestible capsule by methods well-known from the art. In one embodiment, an aqueous solution of the invention may be encapsulated into a digestible capsule.

The encapsulated solution may contain the compound of formula (I) alone, where the compound is dissolved in the DDW of invention, or it may comprise any nutrient or pharmaceutically active ingredient described herein. The amount of the compound of formula (I) may be in the range from 1 to 500 mg per capsule; preferably, from 85 to 500 mg per capsule; more preferably, from 170 to 500 mg per capsule

[00about 90] As used herein, the term “encapsulation” refers to a process to entrap active agents within a carrier material to improve delivery of bioactive molecules into foods. Carrier materials used for design of protective shell of encapsulates must be food-grade, biodegradable and able to form a barrier between the internal phase and its surroundings. Nonexclusive examples of such carrier materials are polysaccharides, proteins, and lipids. The encapsulation process is well-known from the art, see e.g. Nedovich et al, Procedia Food Science 2011, 1: pages 1806-1815.

As used herein, the term “digestible” refers to a material that, when eaten by a subject can be broken down into compounds that can be absorbed and used as nutrients or eliminated by the subject's body. Non-limiting examples of such digestible materials are polysaccharides, proteins, and lipids.

In one embodiment, the aqueous solution of choline succinate of formula (I), as described herein, encapsulated into a digestible capsule is used for enhancing cognitive function in a subject.

As used herein, the term “subject” refers to any mammal including, but are not limited to, human, dog, cat, and horse. In one embodiment, the subject is a human.

The following examples are presented to demonstrate the invention. The examples are illustrative only and are not intended to limit the scope of the invention in any way.

### EXAMPLES

### Example 1

This example demonstrates preparation of water having deuterium content from about 90 to about 135 ppm and ¹H₂¹⁶O isotopologue up to 99.759%.

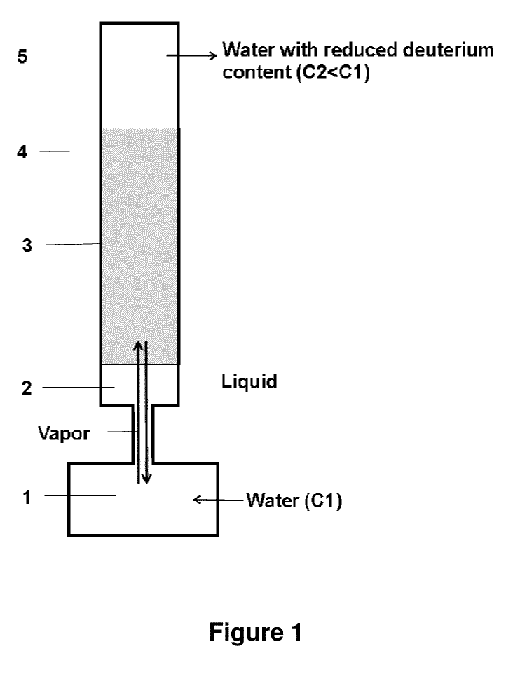

Process: the process includes steps as follows (FIG.1): evaporating the natural water with deuterium content of C1 (155 ppm) in boiling means 1 at 60° C. and pressure 0.2 bars to produce water vapor; supplying the water vapor to the bottom 2 of distillation column 3; carrying out vapor-liquid contact between a descending liquid and an ascending vapor mainly on the surface of the packing 4 within the distillation column; condensing water vapor having reduced deuterium content on condenser 5 installed on upper bound of the distillation column 3; and collecting a part of condensate as condensed water having reduced deuterium content from 10 to about 70 ppm (C2), wherein C2<C1. Finally, water with deuterium content from about 90 to about 135 ppm and ¹H₂¹⁶O isotopologue up to 99.759% is prepared by mixing the water having reduced deuterium content (C2) and the natural water (C1) in certain proportions.

Equipment: the distillation process, when is performed on short distillation columns 3 (e.g. 4-5 m of height), depletes natural water of deuterium-bearing isotopologues (e.g. HOD), while keeps level of H₂¹⁶O equal or less than 99.759%.

Analyses: the deuterium content in resulting water is measured by isotope mass-spectrometry and expressed as deuterium/protium ratio R=D/H, in ppm, where D is the number of deuterium atoms and H is the number of hydrogen atoms.

### Example 2

This example demonstrates a process for preparation of an aqueous solution comprising compound of formula (I) as the nutrient and water having deuterium content from about 90 to about 135 ppm and ¹H₂¹⁶O isotopologue up to 99.759%.

The aqueous solution is prepared by dissolution of compound of formula (I) in water having deuterium content from about 90 to about 135 ppm and ¹H₂¹⁶O isotopologue up to 99.759% (Water_{about 90-135ppm}) in proportions as indicated in Table 1.

TABLE 1

| Ingredient | Content, wt. % |
|---|---|
| Compound of formula (I) | 0.11 |
| Water_{about 90-135 ppm} | 99.89 |

### Example 3

This example demonstrates a process for preparation of beverage comprising compound of formula (I) as the nutrient and water having deuterium content from about 90 to about 135 ppm and ¹H₂¹⁶O isotopologue up to 99.759%.

The beverage is prepared by dissolution of compound of formula (I) in water having deuterium content from about 90 to about 135 ppm and ¹H₂¹⁶O isotopologue up to 99.759% (Water_{about 90-135ppm}) in proportions as indicated in Table 2. The resulted product is bottled in bottles of 330 ml volume.

TABLE 2

| Ingredient | Content, wt. % |
|---|---|
| Compound of formula (I) | 0.1 |
| Water_{about 90-135 ppm} | 99.9 |

This beverage, when is administered orally to a subject for enhancement of cognitive functions in daily amount of 330 ml per serving, provides about 210 mg of choline as the essential nutrient and can be used as a dietary supplement or a medical food for dietary management of cognitive function.

### Example 4

This example demonstrates that beverage, which comprises compound of formula (I) as the nutrient and water having deuterium content from about 90 to about 135 ppm and ¹H₂¹⁶O isotopologue up to 99.759, has an improved taste.

The comparison of tastes of beverages, which were prepared on waters having different deuterium contents, but contained the same amounts of compound of formula (I), was made in blinded experiment. Test beverage was prepared as 0.1% solution of compound of formula (I) in water having deuterium content of 91 ppm and ¹H₂¹⁶O isotopologue up to 99.759% and poured in 7 cups (“Beverage D/H 91 ppm”). Control beverage was prepared as 0.1% solution of compound of formula (I) in water having deuterium content of 150 ppm and ¹H₂¹⁶O isotopologue up to 99.759% and poured in 7 cups (“Beverage D/H 150 ppm, control”). All 14 cups were randomly labeled by the numbers 1 through 14. Another cup was filled with control beverage (“Beverage D/H 150 ppm”) and labeled as “the reference”. During the test procedure, the consumer performs comparison of taste of the reference beverage with taste of samples from cups labeled by numbers from 1 through 14 and rates the difference in taste on scale of −1 (much more unpleasant than the reference) to +1 (much less unpleasant than the reference). A score of 0 indicates the taste of sample is equally as unpleasant as the reference. Results were treated by Mann-Whitney non-parametric analyses. Data are presented in Table 3 as mean±SEM of taste scores.

TABLE 3

| Beverage | Taste scores |
|---|---|
| Beverage D/H 150 ppm (control) | 0.14 ± 0.14 |
| Beverage D/H 91 ppm | 0.72 ± 0.18* |

*Differs significantly of control (P < 0.05)

Table 3 shows that taste of the beverage is significantly much less unpleasant, when this beverage is prepared on water having reduced deuterium content, as compared to the taste of control beverage. Thus, the beverage containing choline succinate salts in amounts effective for ameliorating choline deficiency has the taste acceptable for a consumer, when this beverage is prepared on water from about 90 to about 135 ppm and ¹H₂¹⁶O isotopologue up to 99.759.

### Example 5

This example demonstrates a digestible capsule filled with an aqueous solution of the compound of formula (I) comprising water with the deuterium content from about 90 to about 135 ppm and ¹H₂¹⁶O isotopologue up to 99.759%. Digestible capsules were filled with 50% aqueous solution of the compound of formula (I) in water having deuterium content from about 90 to about 135 ppm and ¹H₂¹⁶O isotopologue up to 99.759% (Water_{about 90-135ppm}) in amounts of from 2 to 1000 mg per capsule.

### Example 6

This example demonstrates that water having deuterium content from about 90 to about 135 ppm and ¹H₂¹⁶O isotopologue less than 99.759% is useful for enhancing cognitive function in adult healthy subjects.

Young adult male Wistar rats were treated with water having D/H ratio 140.7 ppm (control group, n=10) or low deuterium water with D/H ratio about 90.2 ppm (experimental group, n=10) per os as drinking water, 9 days treatment period, ad libitum. Both waters have level of ¹H₂¹⁶O isotopologue less than 99.759%. Control and experimental groups were tested for spatial learning and memory in the modified elevated plus-maze test at day 8^{th} and 9^{th} (acquisition and retention session, respectively), as it has been described by Itoh et al. ltoh et al, Psychopharmacology 19about 90, 101:27-33; ltoh et al, Eur J Pharmacol 1991, 194:71-76. The time for the rat to move from the open arm to the enclosed arm (transfer latency) was used as a parameter of retention and consolidation of memory. Behavioral data were treated by Mann-Whitney non-parametric analyses. Both control and experimental group exhibited significantly decreased transfer latencies on the retention session (TL2) (p<0.05), as compared to the acquisition session (TL1), meaning that rats of both groups remembered the configuration of the open and enclosed arms. However, rats of experimental group exhibited significantly shortened transfer latency on the retention session (TL2), as compared to the control group (p=0.02; Z=2.31). Data are presented in Table 4 as mean±SEM of transfer latencies on days of acquisition session (TL1) and retention session (TL2).

TABLE 4

Transfer latency, s

| Group | TL1 | TL2 |
|---|---|---|
| D/H = 140.7 ppm (control) | 62.0 ± 5.4 | 28.5 ± 5.8 |
| D/H = about 90.2 ppm | 55.6 ± 6.8 | 13.5 ± 2.1* |

*Differs significantly of control TL2 (p < 0.05).

Table 4 shows that administering water having deuterium content from about 90 to about 135 ppm and ¹H₂¹⁶O isotopologue less than 99.759% is useful for enhancing cognitive function in healthy subjects.

### Example 7

This example demonstrates that water having deuterium content from about 90 to about 135 ppm and ¹H₂¹⁶O isotopologue less than 99.759% is useful for enhancing cognitive function in adult healthy subjects.

Three-months-old male C57BI6J mice were treated with water having D/H ratio 153.0 ppm (control group, n=15) or low deuterium waters with D/H ratio 91.7 ppm, 119.9 ppm, and 134.9 ppm (experimental groups, n=15 per group) per os as drinking water, 14 days treatment period, ad libitum. All waters have level of ¹H₂¹⁶O isotopologue less than 99.759%. Control and experimental groups were tested on attention to novel events in the novel cage test. Mice were introduced into a standard plastic cage the size of their home cage filled with small amounts of fresh sawdust. The testing was carried out in a dark quiet room in morning hours. Behavior was videotaped and analyzed by trained observers blind to the treatment protocol. The number of exploratory rearings counted under red light during a 5-min period was used as measure of attention to novel events. Behavioral data were treated by Mann-Whitney non-parametric analyses. Data are presented in Table 5 as mean±SEM of number of exploratory rearings.

TABLE 5

| Group | Number of exploratory rearings |
|---|---|
| D/H = 153.0 ppm (control) | 30.6 ± 1.1 |
| D/H = 134.9 ppm | 33.4 ± 1.6* |
| D/H = 119.9 ppm | 35.4 ± 1.6* |
| D/H = 91.7 ppm | 35.1 ± 1.5* |

*Differs significantly of control (p < 0.05).

Table 5 shows that water having deuterium content from about 90 to about 135 ppm and ¹H₂¹⁶O isotopologue less than 99.759% significantly enhances cognitive function of healthy experimental animals, as compared to the control treatment.

### Example 8

This example demonstrates that water having deuterium content from about 90 to about 135 ppm and ¹H₂¹⁶O isotopologue less than 99.759% is useful for enhancing cognitive function in aged healthy subjects.

Eighteen-months-old male C57BI6J mice were treated with water having D/H ratio 140.7 ppm (control group, n=15) or low deuterium water with D/H ratio 91.6 ppm (experimental group, n=15) per os as drinking water, 14 days treatment period, ad libitum. Both waters have level of ¹H₂¹⁶O isotopologue less than 99.759%. Control and experimental groups were tested for attention to novel events as indicated in the Example 6. Data are presented in Table 6 as mean±SEM of number of exploratory rearings for the first minute of the observations.

TABLE 6

| Group | Number of exploratory rearings |
|---|---|
| D/H = 140.7 ppm (control) | 5.91 ± 0.59 |
| D/H = 91.6 ppm | 8.45 ± 0.83* |

*Differs significantly of control (p < 0.05).

Table 6 shows that water having deuterium content from about 90 to about 135 ppm and ¹H₂¹⁶O isotopologue less than 99.759% significantly enhances cognitive function in healthy aged subjects, as compared to the control treatment.

### Example 9

This example demonstrates that aqueous solution containing compound of formula (I) as the nutrient and water having deuterium content from about 90 to about 135 ppm and ¹H₂¹⁶O isotopologue less than 99.759% is useful for enhancing cognitive function.

Cognitively healthy young adult male Wistar rats weighing 260-280 g were randomly ascribed to groups and supplemented daily with aqueous solution of 5 mg/kg of compound of formula (I) in water of the invention for seven consecutive days. Then, after the break in the supplementation for next seven days, scopolamine 1 mg/kg i.p. or vehicle was administered (at day 14) 30 min before the acquisition trial in the passive avoidance test (“Compound (I)+Scopolamine” group). In “Vehicle+Vehicle” group, rats received vehicle for seven consecutive days and then vehicle 30 min before the acquisition trial in the passive avoidance test at 14^{th} day. In “Vehicle+Scopolamine” group, rats received vehicle for seven consecutive days and then scopolamine 1 mg/kg i.p. 30 min before the acquisition trial in the passive avoidance test at 14th day. Scopolamine is anticholinergic agent that is used commonly to produce acute disturbance of cholinergic neurotransmission, particularly the processes of learning acquisition and short-term memory (Alzheimer's disease-like state). Passive avoidance test requires the rats to behave contrary to their innate tendencies for preference of dark areas and avoidance of bright ones. The apparatus used in this test is composed by a dark compartment and a bright compartment. The latency to enter the dark chamber was measured using 300 s maximum trial duration. In the acquisition trial, the animal received a 0.4 mA electric shock for 3 s when entering the dark chamber. Retention trial was performed 24 h after the acquisition trial. Memory performance is positively correlated with the latency to escape from the bright compartment during retention trials; the greater the latency, the better the recollection. There were no differences between groups in latency to enter the dark chamber during the acquisition trial. However, there were differences in latency to enter the dark chamber during the retention trial, indicating that scopolamine induced disturbance of memory. Data are presented in Table 7 as individual latencies, range, and mean±SEM to enter to the dark compartment during the retention trial.

TABLE 7

| | Groups | | |
|---|---|---|---|
| | | | Aqueous solution of |
| | Vehicle + | Vehicle + | Compound (I) + |
| | Vehicle | Scopolamine | Scopolamine |
| Individual latencies, s | 300 | 300 | 55.2 |
| | 300 | 76 | 282.7 |
| | 300 | 99.8 | 295.5 |
| | 300 | 72.7 | 269.8 |
| | 300 | 300 | 300 |
| | 300 | 186.3 | 300 |
| | 207.3 | 171.4 | 196.8 |
| | 300 | 300 | 300 |
| | 226.7 | 29.1 | 41.4 |
| | 300 | 79.5 | 66 |
| | 300 | | 300 |
| Latencies range | 207.3-300 | 29.1-300 | 41.4-300 |
| Latency mean ± SEM | 284.9 ± 10.2 | 161.5 ± 33.6 | 218.9 ± 33.2 |

Latencies to enter the dark chamber in the retention trial in cognitively healthy rats from group “Vehicle+Vehicle” were accepted as normal ones (207.3-300 s), while a latency less than 207 s, i.e. less than minimal one observed in the cognitively healthy rats, was accepted as disturbance of cognitive function. Rates of disturbance of cognitive function (latency <207 s) were found to be 0.70 and 0.36 for “Vehicle+Scopolamine” and “Compound (I)+Scopolamine” groups, respectively. Relative risk of disturbance of cognitive function in “Compound (I)+Scopolamine” group as compared to “Vehicle+Scopolamine” group was found to be 0.51. Note, the 7-days-break between the last day of the supplementation and the day of onset of scopolamine-induced disturbance of cognitive function completely excludes any effect of the compound of formula (I) on animals in the diseased state, given that the elimination half-life for the compound of formula (I) is less than 1.2 h. Kovalev et al, Exp Clin Pharm 2014, 77(11):23. Thus, administering the aqueous solution of the compound of formula (I) as the nutrient resulted in almost 2-fold reducing the risk of disturbance of cognitive function as compared to the control.

### Example 10

This example demonstrates the use of water with deuterium content from about 90 to about 135 ppm and ¹H₂¹⁶O isotopologue less than 99.759% for preparing the dietary supplement for enhancing cognitive function.

The dietary supplement is prepared by dissolution of calcium chloride, magnesium chloride, and sodium bicarbonate in water having deuterium content within the range of about 90-135 ppm and ¹H₂¹⁶O isotopologue less than 99.759% (Water_{about 90-135ppm}) in proportions as indicated in Table 8. The resulted product is bottled in bottles of 330 ml volume.

TABLE 8

| Ingredient | Content, wt. % |
|---|---|
| Water_{about 90-135 ppm} | 99.953 |
| Calcium Chloride | 0.015 |
| Magnesium Chloride | 0.007 |
| Sodium Bicarbonate | 0.025 |

### Example 11

This example demonstrates the use of water with deuterium content from about 90 to about 135 ppm and ¹H₂¹⁶O isotopologue less than 99.759% for preparing medical food for enhancing cognitive function.

The medical food is prepared by dissolution of calcium chloride, magnesium chloride, and sodium bicarbonate in water having deuterium content within the range of about 90-135 ppm and ¹H₂¹⁶O isotopologue less than 99.759% (Water_{about 90-135ppm}) in proportions as indicated in Table 9. The resulted product is bottled in bottles of 330 ml volume.

TABLE 9

| Ingredient | Content, wt. % |
|---|---|
| Water_{about 90-135 ppm} | 99.953 |
| Calcium Chloride | 0.015 |
| Magnesium Chloride | 0.007 |
| Sodium Bicarbonate | 0.025 |

The medical food is administered orally to a subject at a high risk of dementia in daily amounts from 0.01 to 4 liters for enhancing cognitive function.

### Example 12

This example demonstrates the use of water with deuterium content from about 90 to about 135 ppm and ¹H₂¹⁶O isotopologue less than 99.759% for preparing the pharmaceutical composition for enhancing cognitive function. The pharmaceutical composition for enhancing cognitive function is prepared by dissolution of insulin in water having deuterium content within the range of about 90-135 ppm and ¹H₂¹⁶O isotopologue less than 99.759% (Water_{about 90-135ppm}) in proportions as indicated in Table 10 for unit dosage form.

TABLE 10

| Ingredient | Content |
|---|---|
| Insulin | 20 IU |
| Water_{about 90-135 ppm} | 200 μL |

The composition is administered intranasally to a subject in need thereof once-a-day for enhancing cognitive function.

### Example 13

This example demonstrates an enhancing effect of aqueous solution of compound (I) in water having deuterium content from about 90 to about 135 ppm and ¹H₂¹⁶O isotopologue up to 99.759% on cognitive function in a model of scopolamine-induced amnesia.

Scopolamine-induced amnesia is a widely used model for testing anti-amnestic drugs, which model is considered as relevant to cognitive deficits seen during Alzheimer's disease (AD). Ebert U, Kirch W. Scopolamine model of dementia: electroencephalogram findings and cognitive performance. Eur J Clin Invest. 1998 November; 28(11):944-9.

Passive avoidance (PA) is a classic test for the assessment of efficacy of anti-amnestic drugs in the scopolamine-induced amnesia. Smith C P et al, Pharmacological activity and safety profile of P10358, a novel, orally active acetylcholinesterase inhibitor for Alzheimer's disease. J Pharmacol Exp Ther. 1997 February; 280(2):710-20. Retention latency is a measure of learning and memory in this test. Scopolamine shortens, while anti-amnestic drugs increase, the latency to enter the dark compartment in the memory retention session. A box consisted of a light compartment connected to a dark compartment by a controllable door (Columbus instruments, USA). In the acquisition trial, the rats were individually placed into the light compartment, the door to a dark compartment was opened, and the latency until the rat entered the dark compartment was recorded. After the rat had stepped through the door, the door was closed and an electric shock 0.8 mA was delivered for 3 s via the grid floor. After receiving the foot shock, the rat was returned to a home cage. In the retention trials, each animal was placed into the light compartment, and the step-through latency was recorded until 180 s had elapsed. The effects of treatments on long-term memory performance were assessed in two retention trials, one day (T1) and seven days (T2) after the acquisition trial.

Male adult Wistar rats (280-300 g body weight) were randomly assigned to six groups, by 10 rats per group. Rats in groups 1 and 3 received orally 1 ml of water having deuterium content of 155 ppm; rats in groups 2 and 4 received orally 1 ml of water with deuterium content of about 90 ppm; rats in group 5 received orally 1 ml of aqueous solution of the 10 mg/kg of the compound of formula (I) made on water having deuterium content of 155 ppm; rats in group 6 received orally 1 ml of aqueous solution of the 10 mg/kg of the compound of formula (I) made on water having deuterium content of about 90 ppm; once-a-day for 7 consecutive days. Food and corresponding water were freely available. Then, 30 min before acquisition trial, rats received a single i.p. injection of saline (control groups 1 and 2) or 1 mg/kg scopolamine HBr (groups 3 through 6). The effects of treatments on long-term memory performance were assessed in two retention trials, one day (T1) and seven days (T2) after the acquisition trial. Results are presented in Table 3 as mean±SEM of retention latencies. Data were analyzed for statistical significance by two-way ANOVA with repeated measures followed by Bonferroni's posttest. Differences were considered significant at p<0.05.

TABLE 3

| Group | Retention latency (T1), s | Retention latency (T2), s |
|---|---|---|
| Group 1 | 132.3 ± 16.9 | 135.8 ± 16.2 |
| Group 2 | 134.0 ± 14.24 | 136.9 ± 13.4 |
| Group 3 | 54.8 ± 11.0 | 36.1 ± 5.7 |
| Group 4 | 43.7 ± 8.0 | 34.7 ± 8.4 |
| Group 5 | 79.8 ± 23.8 | 52.0 ± 8.5 |
| Group 6 | 117.4 ± 22.7 | 119.9 ± 11.1 |

Bonferroni's posttest analysis showed that there was no significant difference in retention latencies T1 and T2 between groups 1 and 2 (p>0.05). It means that deuterium content of water did not effect on cognitive performance of intact rats receiving either 155 or about 90 ppm deuterium water.

Bonferroni's posttest analysis showed that scopolamine significantly shortened retention latencies T1 and T2 in groups 3 and 4, as compared to groups 1 (p<0.001) and 2 (p<0.001), respectively. It means that scopolamine induced amnesia in rats receiving 155 ppm as well as about 90 ppm deuterium water. There was no significant difference in retention latencies T1 and T2 between groups 3 and 4 (p>0.05). The last means that water having deuterium content of about 90 ppm did not enhance cognitive function compared to 155 ppm deuterium water in this experimental model.

Bonferroni's posttest analysis showed that there was a statistically significant difference in retention latencies T1 and T2 between group 6 and 4 (p<0.001), while there was no such significant difference between group 5 and 3 (p>0.05). It means that water solution of the compound (I) significantly ameliorated scopolamine-induced amnesia when this solution was made on water having deuterium content of about 90 ppm, while the solution of the same dose of the compound (I) made on water having deuterium content of 155 ppm did not provide any significant anti-amnestic effect. Thus, the aqueous solution of the compound (I) is much more effective when made on water having reduced deuterium content.

Results of this study demonstrating no significant effect of DDW water having content of deuterium about 90 ppm (per se) on cognitive function are somehow in contrary with the suggestion that DDW with content of deuterium less than 90 ppm (i.e. from 89,99 ppm and below) improves cognitive function (RU2338542A2). Without binding to a theory, this may be explained as below:
a cognition enhancing effect of DDW may only be observed when the content of deuterium in DDW is significantly lower than the deuterium content in DDW used in this study. Compared to the experimental data of RU2338542A2, the deuterium content in DDW used in this study is 18 times higher, than it was used in Example 2 of RU2338542A2 (5 ppm). Example 2 of RU2338542 describing the effect of DDW on improvement cognitive function of mice injected with β-amyloid fragment 25-31 refers to water having less than 0.001 mol. % HOD. Given that deuterium in natural water is completely incorporated in HOD isotopologue, 0.001 mol. %, corresponds to the deuterium content 5 ppm;
the experimental cognition model of this study is different from the model of RU2338542A2. As discussed above, both biological effects of DDW (per se) and scale of the biological effects may significantly vary from one experimental model to another (see discussion of Prior Art in [0008]-[0009] of the Background Section of the Specification).
Previously, US2006/0199862A1 showed that a solution of choline succinate (2:1) salt (of formula (i)) in water with the deuterium content of 155 ppm has an enhancing effect on cognitive function in rats with cognitive impairments induced by chronic cerebral ischemia or amyloid injection of a solution of choline succinate (2:1) salt (of formula (i)) in water with the deuterium content of 155 ppm. However, from US2006/0199862A1 it is not possible to presume that the content of deuterium in the water may have an influence on the described effect. Accordingly, the results of this study are surprising in view of US2006/0199862A1 disclosure.

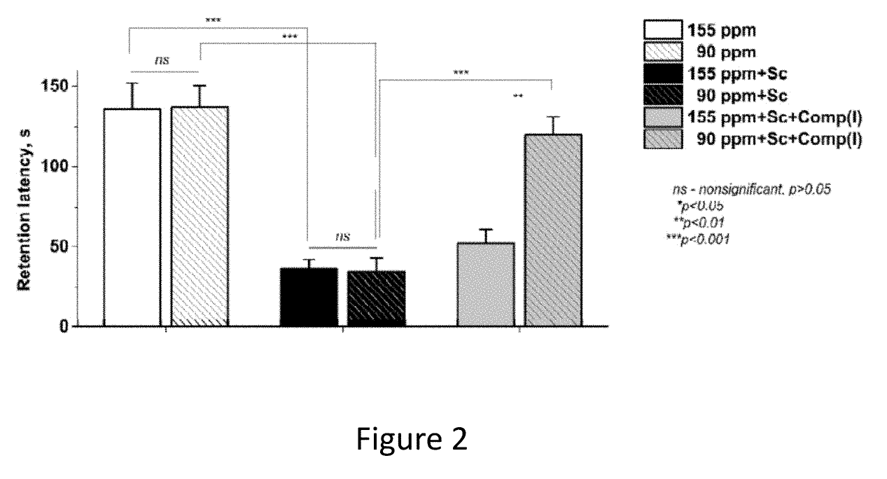

FIG. 2 illustrates the effects of the treatments on retention latencies T2. There was no statistically significant difference (p>0.05) between intact rats receiving water having deuterium content of either about 90 ppm (“about 90 ppm”, i.e. group 2) or 155 ppm deuterium (“155 ppm”, i.e. group 1). There was no statistically significant difference (p>0.05) after the scopolamine injection between rats receiving water having deuterium content of either about 90 ppm (“about 90 ppm+Sc”, i.e. group 4) or 155 ppm deuterium (“155 ppm+Sc”, i.e. group 3). There was a statistically significant difference after the scopolamine injection between rats receiving the aqueous solution of the compound (I) made on water having deuterium content of about 90 ppm (“about 90 ppm+Sc+Comp(I)”, i.e. group 6) and rats receiving the aqueous solution of the compound (I) made on water having deuterium content of 155 ppm (“155 ppm+Sc+Comp(I)”, i.e. group 5). There was a statistically significant difference after the scopolamine injection between rats receiving the aqueous solution of the compound (I) made on water having deuterium content of about 90 ppm (“about 90 ppm+Sc+Comp(I)”, i.e. group 6) and rats receiving only water having deuterium content of about 90 ppm (“about 90 ppm+Sc”, i.e. group 4). There was no statistically significant difference (p>0.05) after the scopolamine injection between rats receiving the aqueous solution of the compound (I) made on water having deuterium content of 155 ppm (“155 ppm+Sc+Comp(I)”, i.e. group 5) and rats receiving only water having deuterium content of 155 ppm (“155 ppm+Sc”, i.e. group 3). Thus, the aqueous solution of the compound (I) made on water having deuterium content of about 90 ppm is significantly more effective for enhancing cognitive function in the experimental model relevant to cognitive deficits seen during Alzheimer's disease than the aqueous solution of the compound (I) made on water having deuterium content of 155 ppm.

1. An aqueous solution comprising the compound of formula (I) 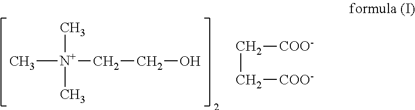
and water, wherein said water has a content of deuterium of from about 90 to about 135 ppm and ¹H₂¹⁶O isotopologue up to 99.759%.

2. The aqueous solution of claim 1, consisting of the compound of formula (I) and water, wherein said water has a content of deuterium of from about 90 to about 135 ppm and ¹H₂¹⁶O isotopologue up to 99.759%.

3. The aqueous solution of claim 1, further comprising a nutrient or a pharmaceutically active compound.
